(19) United States
(12) Patent Application Publication
(10) Pub. No.: US 2019/0026427 A1
(43) Pub. Date: Jan. 24, 2019

(54) GENETIC TESTING AND SUITABILITY SIMULATING SYSTEM

(71) Applicant: Takashi TAKEHARA, Osaka (JP)

(72) Inventor: Takashi TAKEHARA, Osaka (JP)

(21) Appl. No.: 15/757,490

(22) PCT Filed: Sep. 3, 2016
(86) PCT No.: PCT/JP2016/075916

§ 371 Date: Mar. 5, 2018

(30) Foreign Application Priority Data
Sep. 4, 2015 (JP) 2015-175180

(51) Int. Cl. G06F 19/22; C12Q 1/6827; G06F 19/18

(52) U.S. Cl. CPC G06F 19/22; C12Q 2600/156; G06F 19/18; C12Q 1/6827

(57) ABSTRACT

Provided is a genetic testing and suitability simulating system that, by testing the DNA of a user and comparing the user's DNA to the DNA information of a distinguished athlete, simulates, from a comparative perspective, the potential suitability of the user in a given type of sport and in a given position in a specific sporting activity and also enables continuous support in the user's training.

### TECHNICAL FIELD

The present invention relates to a genetic testing and suitability simulation system that is capable of testing DNA of a user and comparing the tested DNA of the user with DNA information of well-known athletes, whereby it is possible to simulate the potential suitability of the user for specific sports or specific positions in a given sport game from a relative viewpoint and furthermore to continuously support training of the user.

### BACKGROUND ART

Description of Consideration

At the present time, a wide range of age groups is enjoying a variety of sports all over the world as hobbies and for health maintenance, and many people are undergoing training diligently everyday as a part of life and finding life more enjoyable through participation in sports. It is considered that sports are integrated into culture and education, in addition to simple body control and game enjoyment, whereby sports are creating a way of life based on pleasure experienced during effort, on educational values such as setting a good example, and on respect of universal, fundamental, and ethical principles.

In addition, with regard to the Olympics, which is the world's largest sports festival, it has been said that the health and morality of global citizens are improved through sports, that the degree of understanding is deepened through mutual communication and that a circle of friendship is widened, whereby a livable society is made, and that sports contribute to the establishment and maintenance of world peace. Sports are considered to greatly affect connections between people and world peace, and are actively incorporated in the education of children.

Particularly in advanced nations, children from childhood to young adulthood actively learn sports as a part of education, and have opportunities to participate in sport clubs that are operated by schools or other entities. For this reason, children follow well-known athletes in sports in which they participate, and, in many cases, undergo training while dreaming of playing in the same professional sports as the well-known athletes. It is common for parents to actively support the training of their children from the aspect of facilities, tools, and environments.

In recent years, it has become possible to recognize the potential exercise capacity of each person by testing genetic information of the human body that each person has by nature (simply referred to as “DNA” in this specification). It is possible to distinguish between personal characteristics (talent and physical conditions), such as muscular force, endurance, instantaneous force, energy metabolism efficiency, and the lipid metabolic rate, through genetic testing based on DNA. For example, see Patent Document 1 (Japanese Patent Application Publication No. 2005-538710).

According to the DNA testing described above, it is possible for an athlete to recognize whether he/she is suitable for sports that require instantaneous force or is suitable for sports that require endurance. Furthermore, it is possible to recognize whether his/her metabolic type is a visceral fat type or a subcutaneous fat type, whereby it is possible for each athlete to introduce a high-efficiency custom-made training method that coincides with his/her potential suitability and to easily adopt a dietary practice that is suitable for health maintenance and body building. Additionally, if his/her potential suitability is recognized earlier, it is possible for each athlete to start training in a sport that coincides with his/her suitability. Consequently, it is very effective for children from childhood to young adulthood, who actively work on sports, to recognize their potential suitability through DNA testing.

### RELATED ART DOCUMENT

### Patent Document

Patent Document 1: Japanese Patent Application Publication No. 2005-538710

### DISCLOSURE OF THE INVENTION

### Problem to be Solved by the Invention

However, in a conventional DNA testing system for testing potential suitability for sports, absolute potential suitability is tested based on past knowledge, whereby it is not possible to perform a relative comparison with other people or to continuously support training in sports after testing. For this reason, it is necessary for an athlete to select and decide the optimum training menu based entirely on his/her potential suitability recognized through DNA testing. After testing, it is not possible to acquire information related to DNA. If sports are a continuous self-improvement method for mind and body training, it is considered that continuity is urgently needed for a system and service for supporting athletes.

The present invention has been made in view of the above problems, and it is an object of the present invention to provide a genetic testing and suitability simulation system that is capable of testing the DNA of a user and comparing the tested DNA of the user with DNA information of well-known athletes, whereby it is possible to simulate the potential suitability of the user for specific sports or specific positions in a given sport game from a relative viewpoint and furthermore to continuously support the training of the user.

### Meaning for Solving the Problem

In accordance with an embodiment of the present invention, there is provided a genetic testing and suitability simulation system that is capable of analyzing a gene from DNA of a user and simulating potential the suitability of the user, the genetic testing and suitability simulation system including:

sampling a cell of the user on which DNA analysis is possible (Step 1);

analyzing DNA arrangement information from the cell sampled at Step 1 (Step 2);

performing genetic analysis from the DNA arrangement information to identify a genetic type (Step 3);

selecting a genetic type suitable for an arbitrary sport motion from an arbitrary genetic polymorphism (Step 4);

comparing the genetic type of the user identified through the genetic analysis with the genetic type selected at Step 4 (Step 5);

calculating a degree of the user's suitability for the arbitrary sport motion from a result of the comparison (Step 6);

determining whether the user is suitable for the arbitrary sport motion from the degree of suitability calculated at Step 6 (Step 7); and

presenting a result of calculation at Step 6 and a result of determination at Step 7 to the user as a result of suitability for the arbitrary sport motion through a device possessed by the user (Step 8).

In accordance with another embodiment of the present invention, there is provided a genetic testing and suitability simulation system that is capable of analyzing a gene from the DNA of a user and simulating the potential suitability of the user, the genetic testing and suitability simulation system including:

sampling a cell of the user on which DNA analysis is possible (Step 1);

analyzing DNA arrangement information from the cell sampled at Step 1 (Step 2);

performing genetic analysis from the DNA arrangement information to identify a genetic type (Step 3);

selecting a genetic polymorphism related to an arbitrary sport motion (Step 4);

selecting an arbitrary genetic type from the genetic polymorphism selected at Step 4 (Step 5);

comparing the genetic type of the user identified through the genetic analysis at Step 3 with the genetic type selected from the genetic polymorphism related to the arbitrary sport motion at Step 5 (Step 6);

calculating a degree of the user's suitability for the arbitrary sport motion from a result of the comparison (Step 7);

determining whether the user is suitable for the arbitrary sport motion from the degree of suitability calculated at Step 7 (Step 8); and

presenting a result of calculation at Step 7 and a result of determination at Step 8 to the user as a result of suitability for the arbitrary sport motion through a device possessed by the user (Step 9).

In accordance with a further embodiment of the present invention, there is provided a genetic testing and suitability simulation system that is capable of analyzing a gene from the DNA of a user and simulating the potential suitability of the user, the genetic testing and suitability simulation system including:

sampling a cell of the user on which DNA analysis is possible (Step 1);

analyzing DNA arrangement information from the cell sampled at Step 1 (Step 2);

performing genetic analysis from the DNA arrangement information to identify a genetic type (Step 3);

selecting a genetic type suitable for an arbitrary game motion from an arbitrary genetic polymorphism (Step 4);

comparing the genetic type of the user identified through the genetic analysis at Step 3 with the genetic type suitable for the arbitrary game motion selected at Step 4 (Step 5);

calculating a degree of the user's suitability for the arbitrary game motion from a result of the comparison (Step 6);

determining whether the user is suitable for the arbitrary game motion from the degree of suitability calculated at Step 6 (Step 7); and

presenting a result of the calculation at Step 6 and a result of the determination at Step 7 to the user as a result of suitability for the arbitrary game motion through a device possessed by the user (Step 8).

The sampling of the cell of the user, the analysis of the DNA arrangement information, the analysis of the gene, and the identification of the genetic type are consistently performed, and then the genetic type of the user is compared with a genetic polymorphism that is suitable for a predetermined sport motion and game motion. Here, the compared genetic type includes a genetic type that directly affects sports, such as endurance, speed, and instantaneous force, and a genetic type that has information about respective parts constituting the body. In addition, the compared genetic type includes a genetic type that has mental information, such as the ability to think, the power of attention, and sociability. A plurality of genetic types that are set in advance depending on respective game motions is set as a game motion. The result of comparison in sport motions shows a stepwise degree of suitability for each of the compared genetic types, for example, a four-step degree of suitability for each of the compared genetic types, and evaluates the suitability of the user for each of the sport motions that are related to the respective genetic types. Furthermore, in the game motion, a stepwise degree of suitability, for example, a four-step degree of suitability, is given for each of a plurality of genetic types that are compared, and this degree of suitability is compared with a degree of suitability for a plurality of genetic types that are set in advance depending on respective game motions, whereby the evaluation of suitability is performed. In addition, the result of the evaluation is transmitted to a device possessed by the user via the Internet, whereby it is possible to rapidly and correctly present the result of the evaluation. In addition, it is possible to continuously provide precise training advices and a variety of supports depending on the potential ability of the user.

The genetic testing and suitability simulation system according to the present invention having the features described above may further include presenting a role of the user in an arbitrary sport from the result of suitability for the arbitrary sport motion and the result of suitability for the arbitrary game motion.

After suitability for a variety of sport motions and game motions is evaluated from the genetic type of the user, the result of suitability for each of the sport motions and each of the game motions is comprehensively evaluated, whereby it is possible to evaluate which game the user is suitable for and, furthermore, to evaluate suitability for a particular role (a position) of the user in a given game.

### BRIEF DESCRIPTION OF THE DRAWINGS

FIG. 1 is a schematic view showing the scheme of a genetic testing and suitability simulation system according to the present invention;

FIG. 2 is a schematic view showing an example of the construction of the genetic testing and suitability simulation system according to the present invention;

FIG. 3 is a block diagram showing information processing in which an application is installed in a user terminal;

FIG. 4 is a block diagram showing information processing in which respective information transmitted from a manager terminal 11 is stored in corresponding databases of a DNA simulation server 1;

FIG. 5 is a block diagram showing information processing in which the DNA simulation server delivers content information to the user terminal;

FIG. 6 is a block diagram showing information processing in which the DNA simulation server performs suitability simulation using DNA information of a user; and

FIG. 7 is a block diagram showing a concrete procedure of DNA testing and comparison in DNA information using the genetic testing and suitability simulation system according to the present invention.

### BEST MODE FOR CARRYING OUT THE INVENTION

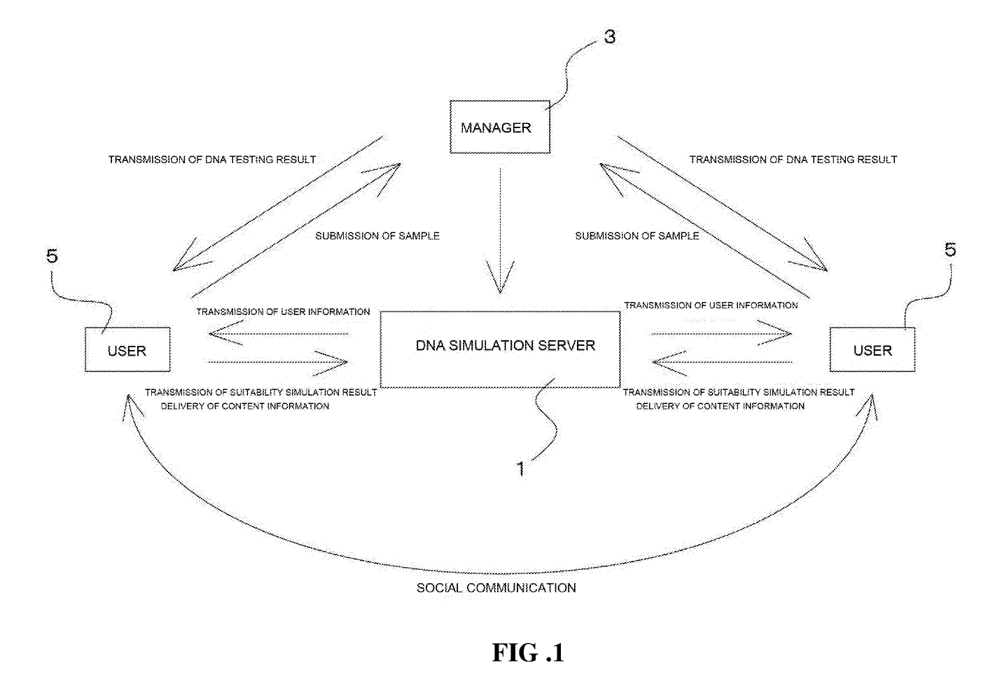

Hereinafter, exemplary embodiments of a genetic testing and suitability simulation system according to the present invention will be described in detail with reference to FIGS. 1 to 7. However, the present invention is not limited to the illustrated embodiments. In addition, the drawings are provided to conceptually describe the present invention. For easy comprehension, therefore, dimensions, ratios, or numbers may be exaggerated or simplified as needed. Furthermore, in the following description, the same reference numbers will be used to refer to the same or like parts, a duplicate description of which will be omitted.

First, the concept of a genetic testing and suitability simulation system according to the present invention will be described with reference to FIG. 1. FIG. 1 is a schematic view showing the scheme of a genetic testing and suitability simulation system according to the present invention. The genetic testing and suitability simulation system according to the present invention generally includes a DNA simulation server 1, a manager 3, and a user 5.

In order to use the genetic testing and suitability simulation system according to the present invention, first, the user obtains and submits a sample of an oral mucous membrane, hair, etc. necessary for DNA testing to the manager in accordance with a DNA testing method and procedure set by the manager. It is preferable for the manager 3 to set the DNA testing method and procedure as simple and convenient as possible such that the genetic testing and suitability simulation system according to the present invention can be operated surely and rapidly.

After receiving a cell sample for DNA testing and basic quantitative information, a description of which will follow, from the user 5, the manager 3 tests the DNA of the user 5 or requests a corporation that is capable of performing DNA testing to test the DNA of the user 5. The manager 3 notifies the user 5 about DNA information of the user 5 acquired through the DNA testing in the form of electronic information or by means of paper. At the same time, the manager 3 records user information, a description of which will follow, the DNA information, and the basic quantitative information in the DNA simulation server 1 using a manager terminal 11. Upon receiving the DNA information of the user 5 from the manager 3, the DNA simulation server 1 performs various suitability simulation on the DNA information of the user 5 using a system, which constitutes the inner part of the DNA simulation server 1 and which will be described later, and transmits the results of simulation to a user terminal 9 possessed by the user 5, such as a smartphone or a tablet PC, via the Internet 13 (or a mobile telephone communication network). In addition, the DNA simulation server 1 stores the results of simulation in a database unit of the DNA simulation server 1. The results of the simulation are used as comparative data, which will be used in subsequent simulation.

It is possible for the user 5 to download and install an application 17, delivered in advance by the manager 3, in a user terminal 9, to read information received from the DNA simulation server 1 using the application 17, and to use the content.

The application 17 includes a personalized page for performing basic manipulation and an athlete diary page for allowing a user to manage a schedule related to sports, such as training, as basic content. Furthermore, additional paid content may be used to read the results of the suitability simulation delivered by the DNA simulation server 1. The user 5 may compare his/her DNA information with DNA information of a plurality of well-known athletes in order to acquire information about which sports the user is suitable for based on similarities and differences, and furthermore, information about which position the user is suitable for in a specific sport game.

In addition, the user 5 may use the application 17 to download and use a variety of content information delivered by the DNA simulation server 1 depending on a charging status. Concrete content information includes text, images, sound, video, or a combination thereof. For example, the content information may include video that introduces training menus and techniques written by well-known athletes. Furthermore, the content information may include a correspondence course based on text and video. It is possible for the user 5 to perform new training based on downloaded content. Furthermore, it is possible for the user 5 to purchase articles related to sports from an Internet marketplace that operates the system according to the present invention.

Furthermore, the user 5 transmits problems and questions that occur during daily training to the DNA simulation server 1 using the application 17. On the end of the manager 3, a professional trainer replies to provide guidance (an assembly of a spot training method and a medium- and long-term curriculum) to lead the user 5 to the solution to the problems and questions.

Furthermore, the genetic testing and suitability simulation system according to the present invention includes a social networking service for allowing users who use the system to communicate with each other. For example, a notice board that can be written and read only by users who use the system may be provided, and the users may transmit and receive messages to and from each other. Between the users who use the genetic testing and suitability simulation system according to the present invention, people who play the same kind of sports may exchange a variety of information, whereby it is possible to constantly maintain a spirit of competition and to enhance motivation.

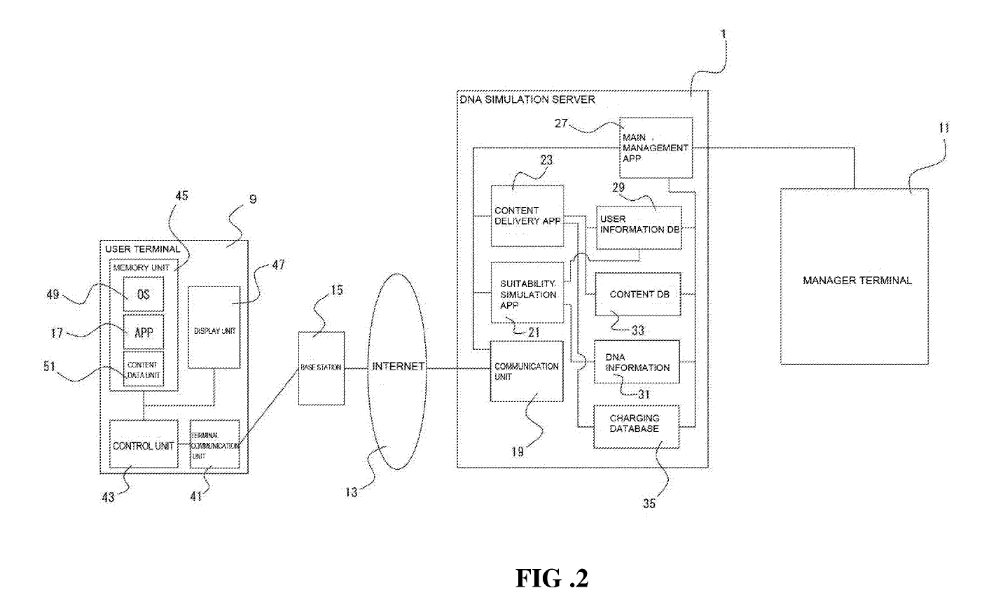

Next, the genetic testing and suitability simulation system according to the present invention will be described in more detail with reference to FIG. 2. FIG. 2 is a schematic view showing an example of the construction of the genetic testing and suitability simulation system according to the present invention. Terminals constituting the genetic testing and suitability simulation system according to the present invention include a DNA simulation server 1, a user terminal 9, and a manager terminal 11. The DNA simulation server 1 and the manager terminal 11 may be connected to each other through a local area network in a wired fashion or via the Internet 13. The DNA simulation server 1 and the user terminal 9 may be connected to each other via the Internet 13 (or a mobile phone communication network). Furthermore, the DNA simulation server 1 and the user terminal 9 may perform information communication via a base station 15. Here, the information communication between the DNA simulation server 1 and the user terminal 9 is performed, for example, based on 3G or 4G communication technology. The base station 15 may be replaced by a so-called access point or a wireless router in order to achieve wireless connection. In the case in which a personal computer is present, the user terminal 9 may be connected to the DNA simulation server 1 via a wired or wireless local area network.

The DNA simulation server 1 includes an application unit for managing and calculating a variety of data and for delivering content and performing simulation, a database unit for storing a variety of data, and a communication unit 19. The application unit is provided with a suitability simulation application 21, a content delivery application 23, and a main management application 27. The database unit is provided with a user information database 29, a DNA information database 31, a content database 33, and a charging database 35. However, a plurality of physically separate devices is not necessarily needed in order to perform the above functions. A single device may provide all of the functions.

The user terminal 9 is basically a multifunctional mobile terminal (hereinafter, also simply referred to as a “smartphone” in this specification) and a tablet PC. The user terminal 9 is provided with at least a terminal communication unit 41, a control unit 43, a memory unit 45, and a display unit 47. In addition, the memory unit 45 stores an operation system 49 (hereinafter, also simply referred to as an “OS” in this specification), and the user terminal 9 is controlled by the control unit 43, which functions as a processor.

Furthermore, it is possible for the memory unit 45 to store an application 17 for managing information communication with the DNA simulation server 1 and operating the genetic testing and suitability simulation system according to the present invention and to store content information received from the DNA simulation server 1, in addition to the OS 49. However, the memory unit 45 is not limited to a general flash memory. The OS 49 may be stored in a memory provided in the control unit 43. The content information may include text data, a plurality of sound data, video data, or a combination of sound data and video data.

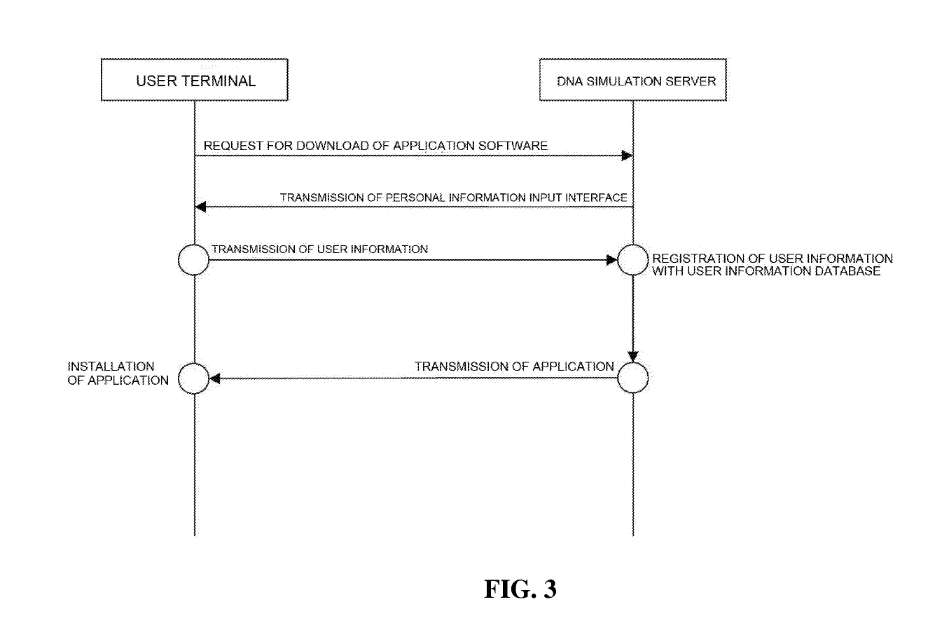

Next, the flow of information processing in which the application 17 is installed in the user terminal 9 will be described with reference to FIG. 3. FIG. 3 is a block diagram showing information processing in which the application 17 is installed in the user terminal 9. In order to use the genetic testing and suitability simulation system according to the present invention, it is necessary to install the application in the user terminal 9 possessed by the user 5 in advance. The user accesses the DNA simulation server 1 via the Internet 13 through the base station 15 from the user terminal 9, and downloads and installs the application 17 from a web server (not shown) or a web site that also serves as the web server and provides the content delivery application 23.

At the same time, the user registers the user terminal 9 with the DNA simulation server 1 from an interface screen of the web site. In addition, the user transmits personal information, such as his/her sex, age, occupation, residential area, working area, hobbies, and interest, to the DNA simulation server 1. Furthermore, the user transmits personal information, such as the kind of sports that he/she plays and his/her position in the sports, to the DNA simulation server 1. At this time, information is exchanged using asynchronous communication. Consequently, it is not necessary for both the user terminal 9 and the DNA simulation server 1 to be in an on line state. Even when one of the user terminal 9 and the DNA simulation server 1 is temporarily in an off line state, it is possible to complete transmission and reception.

The DNA simulation server 1 stores personal information of the user in the user information database 29 such that the user is related to the registered user terminal 9. The personal information includes charging information for using the genetic testing and suitability simulation system according to the present invention. At an early stage immediately after installation, however, the charging information is 0.

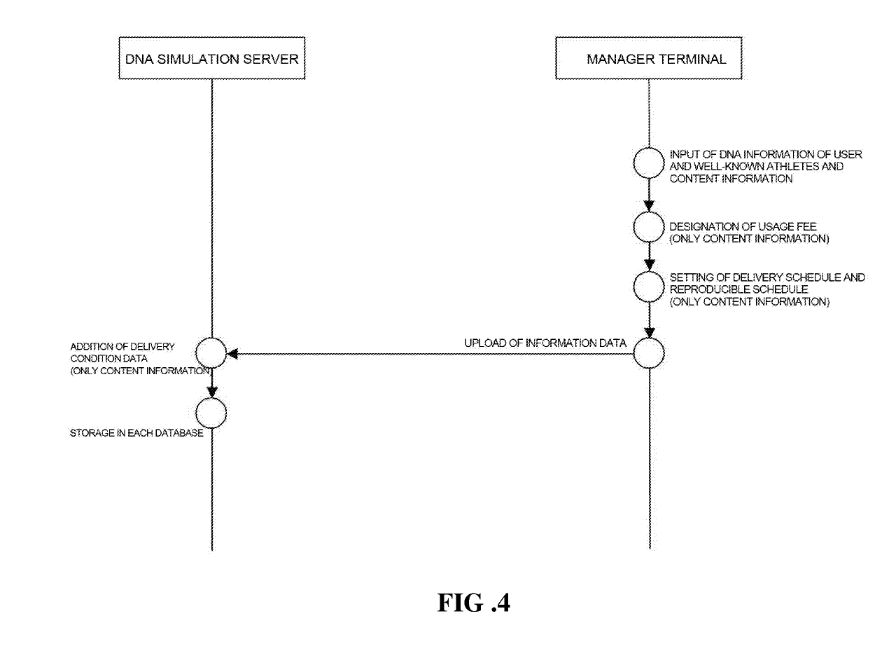

Next, the flow in which the DNA information of the user transmitted from the manager terminal 11 to the DNA simulation server 1, DNA information of well-known athletes, and content information are stored in the database unit provided in the DNA simulation server 1 will be described with reference to FIG. 4. FIG. 4 is a block diagram showing information processing in which respective information transmitted from the manager terminal 11 is stored in corresponding databases of the DNA simulation server 1.

The manager 3 accesses the DNA simulation server 1 via the local area network or the Internet 13 from the manager terminal 11, and manipulates an interface screen that is provided by the main management application 27 in order to transmit desired information to the main management application 27 provided in the DNA simulation server 1.

In addition, the content information is not delivered to all users, but the content information is delivered to each user depending on the charging status of the user. Consequently, it is necessary to set delivery conditions including use charge necessary to deliver each piece of content information, a schedule for delivering the content information from the content delivery application 23 to the user terminal, and a schedule for reproducing the content information in the user terminal 9.

In the case in which the manager 3 transmits the content information to the DNA simulation server 1, therefore, the manager 3 accesses the DNA simulation server 1 via the local area network or the Internet 13 from the manager terminal 11. Subsequently, the manager 3 manipulates the interface screen that is provided by the main management application 27 in order to designate a user that transmits each piece 3 of content information and to set a schedule. In addition, the manager 3 transmits the information to the main management application 27 provided in the DNA simulation server 1.

The main management application 27 stores DNA information of a user uploaded by the manager 3, DNA information of well-known athletes, and content information in the database unit. More specifically, the DNA information of the user is stored in the user information database 29, the DNA information of the well-known athletes is stored in the DNA information database 31, and the content information is stored in the content database 33 in the state in which the setting of a schedule for delivering each piece of content information set by the manager 3 from the content delivery application 23 to the user terminal 9 and the setting of a schedule for reproducing the content information received by the user terminal 9 are added as delivery condition data. Meanwhile, the manager 3 may access the main management application 27 in order to change the delivery condition data as needed.

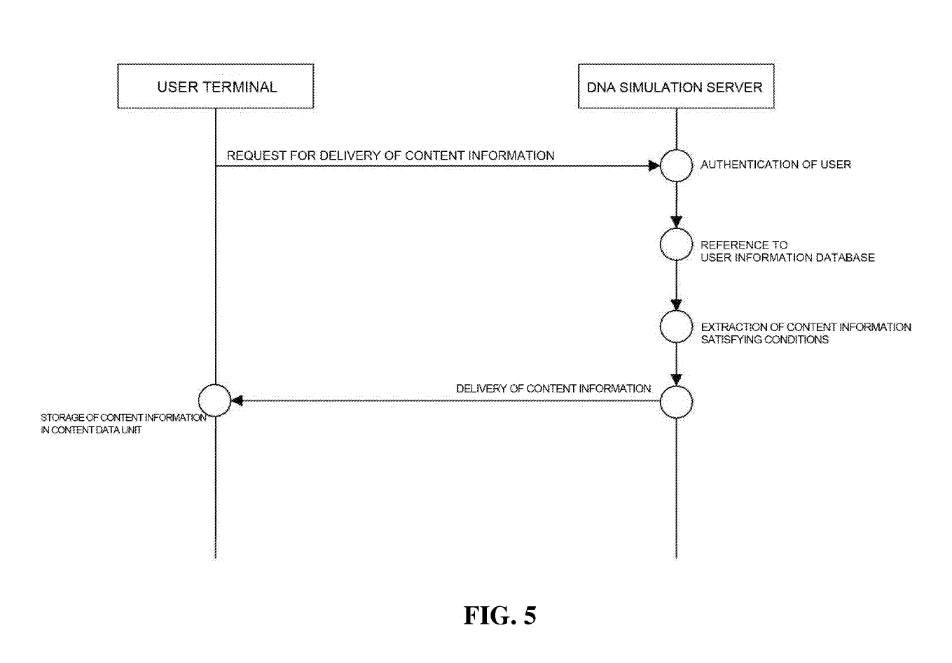

Next, the flow of information processing in which the DNA simulation server 1 receives a request for delivering content information from the user terminal 9 and delivers the content information to the user terminal 9 will be described with reference to FIG. 5. FIG. 5 is a block diagram showing information processing in which the DNA simulation server 1 delivers content information to the user terminal 9. When the DNA simulation server 1 receives a request for delivering content information from the user terminal 9, the content delivery application 23 receives the request via the communication unit 19 and compares the user information with personal information in the user information database 29 to determine whether the user is an authorized user. After that, the charging database is accessed to check the charging status of the user. Subsequently, the content delivery application 23 retrieves the usage status of the user in the user information database 29 and extracts the personal information of the user, the usage status of the user, and content information coinciding with the user's request for delivery with reference to the content database 33. For example, individual charging or usage-based charging conditions are provided, and when the charging status of the user is not satisfied, the content information is not extracted.

Subsequently, the content delivery application 23 delivers the extracted content information to the user terminal 9 via the Internet 13 from the communication unit 19.

The user terminal 9 stores the received content information in a content data unit 51 provided in the memory unit 45. In the case in which the information storage capacity of the user terminal 9 is not sufficient, however, old content information is overwritten with new content information. Alternatively, the information stored in the user terminal 9 is arranged from the display unit 47 in order to store new content information.

It is possible for the user to read and listen to the content information stored in his/her user terminal 9 as described above by manipulating an interface that the application 17 displays on the display unit 47 using the application 17 provided in the memory unit 45.

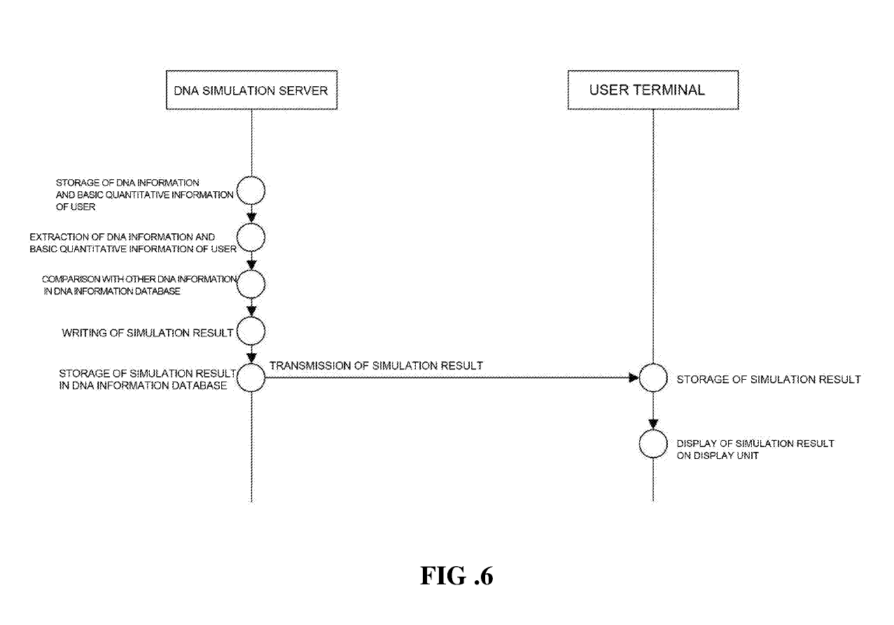

Next, the flow of information processing in which the DNA simulation server 1 compares the DNA information of the user with DNA information of well-known athletes will be described with reference to FIG. 6. FIG. 6 is a block diagram showing the process in which the DNA simulation server 1 performs suitability simulation using the DNA information of the user.

First, the main management application 27 provided in the DNA simulation server 1 receives the DNA information and the basic quantitative information of the user from the manager 3 and then stores the received information in the user information database 29. Subsequently, in the case in which the charging status of the user satisfies the suitability simulation usage conditions and in which the user wishes suitability simulation, the suitability simulation application 21 provided in the DNA simulation server 1 extracts the DNA information of the user stored in the user information database 29 and compares the extracted DNA information of the user with the DNA information of the well-known athletes stored in the DNA information database 31.

The suitability simulation application 21 writes out the result of simulation based on the comparison of DNA information as information, and stores the result of simulation in the DNA information database 31 provided in the DNA simulation server 1. At this time, it is preferable for information about the result of simulation to be stored in the state of being linked with the personal information of the user stored in the user information database 29 such that it is possible to extract the information about the result of simulation even via the user information database 29.

The result of simulation is stored and accumulated in the DNA information database 31 every time such that the amount of comparative data is increased at the time of suitability simulation. As the number of times the suitability simulation is performed is increased, it is possible to perform more accurate simulation. This is one of the characteristics that indicate the superiority of the genetic testing and suitability simulation system according to the present invention.

At the same time, the written information about the result of simulation is transmitted to the user terminal 9. The suitability simulation application 21 transmits the information about the result of simulation to the communication unit 19. Subsequently, the suitability simulation application 21 delivers the information about the result of simulation to the user terminal 9 via the Internet 13 from the communication unit 19.

The user terminal 9 stores the received information about the result of simulation in the content data unit 51 provided in the memory unit 45. It is possible for the user to read the information about the result of simulation stored in his/her user terminal 9 in accordance with the above flow by manipulating an interface that the application 17 reflects to the display unit 47 using the application 17 provided in the memory unit 45.

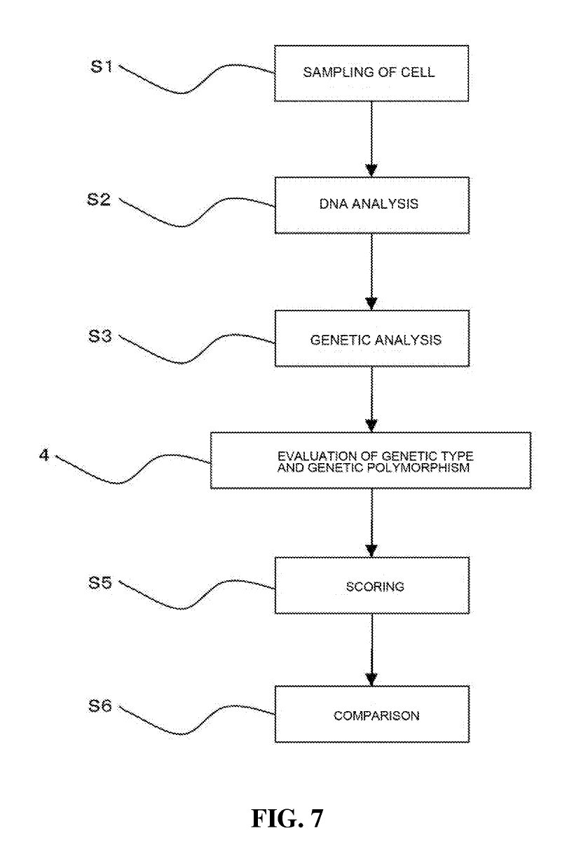

Next, a concrete DNA information comparison method will be described in detail with reference to FIG. 7. FIG. 7 is a block diagram showing a concrete procedure of DNA testing and comparison in DNA information using the genetic testing and suitability simulation system according to the present invention. First, a cell sample necessary to acquire DNA information is obtained from a user (S1). It is preferable to use hair of the user or an oral mucous membrane and a nasal mucous membrane, which can be easily obtained, as the cell sample.

Subsequently, DNA is extracted from the cell sample obtained from the user using a nucleic acid extractor, and then the DNA is amplified using a nucleic acid amplifier in order to analyze the arrangement of the DNA (S2). After the arrangement of the DNA has been analyzed, a plurality of genetic types and genetic polymorphisms related to the ability of a predetermined sport are analyzed (S3). Here, the analyzed genetic types and genetic polymorphisms include genetic types and genetic polymorphisms that directly affect sports, such as endurance, speed, and instantaneous force, and genetic types and genetic polymorphisms that have information about respective parts constituting the body. In addition, the analyzed genetic types and genetic polymorphisms include genetic types and genetic polymorphisms that pertain to mental information, such as the ability to think, the power of attention, and sociability.

Subsequently, the genetic types and the genetic polymorphisms analyzed at the previous step are evaluated (in stages) (S4), and then a score is given for each of the genetic types and the genetic polymorphisms according to the result of the evaluation, and the score of each of the evaluated genetic types and genetic polymorphisms and scores obtained by adding all of the evaluated genetic types and genetic polymorphisms together are digitized so as to be individually handled (S5).

After all of the above five steps have been completed, the score of each of the evaluated genetic types and genetic polymorphisms and the scores obtained by adding all of the evaluated genetic types and genetic polymorphisms together are compared with scores acquired through evaluation of genetic types and genetic polymorphisms of the well-known athletes stored in the DNA information database 31 (S6).

At the comparison performed at step S6, comparison between the scores of the genetic types and the genetic polymorphisms is important, and the comparison is performed to recognize the potential of the user in the sports. It is possible to simulate the genre of the sports and, furthermore, suitability for a position in a specific sport game, through the comparison with the scores acquired by evaluating the genetic types and the genetic polymorphisms of a plurality of well-known athletes.

Next, the flow of information processing occurring between the user terminal 9 and the DNA simulation server 1 in charging related to content that is desired by the user will be described. When the user uses paid content provided by the DNA simulation server 1, as described above, it is necessary for the user to pay a usage fee set by the manager 3. In the paid content, individual charging or usage-based charging is set for each piece of content information.

First, the user manipulates an interface constituted by the application 17 of the user terminal 9 to select desired content information from among a variety of content information provided by the genetic testing and suitability simulation system according to the present invention and to transmit the desired content information to the DNA simulation server 1. The request for the desired content information passes through the communication unit 19 of the DNA simulation server 1 via the Internet 13 through the base station 15 from the terminal communication unit 41 of the user terminal 9, and then reaches the content delivery application 23.

The content delivery application 23 checks the request for the desired content information received from the user terminal 9, and checks a usage fee necessary to retrieve the content database 33 and to deliver the content information. Subsequently, the content delivery application 23 checks the user information database 29, authenticates the user who transmitted the request for the desired content information, and checks the charging status of the user stored in the charging database 35.

In the case in which the charging status of the user satisfies content information delivery conditions according to the request for the desired content information, the content delivery application 23 directly extracts the content information from the content database 33, and delivers the extracted content information to the user terminal 9. In the case in which the charging status of the user does not satisfy content information delivery conditions according to the request for the desired content information, the user terminal 9 is informed of a usage fee necessary to acquire the content information, and suspension of delivery of the content information is recorded in the user information database 29.

The notification of the usage fee necessary to acquire the content information is transmitted to the user terminal 9 through the base station 15 via the Internet 13 from the communication unit 19. The user terminal 9 transmits the notification received by the terminal communication unit 43 to the application 17. The application 17 is displayed on the display unit 47 in order to inform the user of the notification. The user pays the usage fee necessary to acquire the content information to the manager 3.

As a method of the user paying the usage fee to the manager 3, the user may pay the usage fee to the manager 3 in cash. In this case, however, the procedure for payment and reception is complicated. For this reason, it is suitable to pay the usage fee in electronic money, such as credit card payment, Google Wallet (registered trademark), or PayPal (registered trademark).

The manager 3 confirms the usage fee necessary to acquire the content information, remitted by the user, and directly manipulates the manager terminal 11 to transmit information about the user and the amount of money to the main management application 27. The main management application 27 identifies the user notified from the manager terminal 11 with reference to the user information database 29, and then adds the amount of money received from the manager terminal 11 to the charging history of the user stored in the charging database 35 in order to update the charging status of the user.

Subsequently, the main management application 27 notifies the content delivery application 23 of the fact that the charging status of the user has been updated. Upon receiving notification of the fact that the charging status of the user has been updated from the main management application 27, the content delivery application 23 confirms the usage fee necessary to deliver the suspended content information with reference to the suspension of delivering the content information recorded in the user information database 29, and determines whether the charging status of the user satisfies the content information delivery conditions with reference to the charging database 35.

In the case in which the charging status of the user satisfies the content information delivery conditions, the content delivery application 23 extracts the content information from the content database 33, and delivers the extracted content information to the user terminal. In addition, the content delivery application 23 stores the delivery history of the delivered content information in the content database 33. The content information is delivered to the terminal communication unit 41 through the base station 15 via the Internet 13 from the communication unit 19. The user terminal 9 stores the content information received by the terminal communication unit 41 in the content data unit 51. The user may manipulate the interface constituted by the application 17 in order to display and reproduce the content information through the display unit 47 and a speaker (not shown) mounted in the user terminal 9.

It is possible for the manager 3 to successively confirm the charging status of each user stored in the charging database 35 and the delivery history of each piece of content information stored in the content database 33 via the main management application 27 using the manager terminal 11. In addition, the main management application 27 may automatically prepare, for example, a monthly report stating the delivery history of each piece of content information and the charging status of each user on the last day of each month, and may transmit the report to the manager terminal 11 by electronic mail. Alternatively, content information that is less desired to deliver may be picked up, and electronic mail for urging update of the content information may be transmitted to the manager 3 as needed.

In addition, the manager 3 may publish advertisements of sponsor companies or partner companies on the interface constituted by the application 17 of the user terminal 9, an Internet marketplace, etc. in order to acquire advertisement rates.

As described above, the genetic suitability simulation system using the genetic testing and suitability simulation system according to the present invention is capable of providing training guidance to a user based on scientific grounds, compared to a conventional system. In addition, the genetic suitability simulation system is capable of further adding to the value of established well-known athletes and retired well-known athletes and creating new jobs through the provision of DNA information and the cooperative manufacture of various kinds of content information.

Although the embodiments of the present invention have been described with reference to the drawings, the present invention is not limited to the embodiments, and those skilled in the art will appreciate that other improvements and modifications are possible, without departing from the scope and spirit of the invention as disclosed in the accompanying claims.

### INDUSTRIAL APPLICABILITY

According to the present invention, the DNA of the user 5 is tested and compared with the DNA information of the well-known athletes, whereby it is possible to simulate the potential suitability of the user for specific sports or specific positions in a given sport game and furthermore to continuously support training of the user.

### DESCRIPTION OF REFERENCE NUMERALS

1 DNA simulation server
3 Manager
5 User
9 User terminal
11 Manager terminal
13 Interface
15 Base station
17 Application
19 Communication unit
21 Suitability simulation application
23 Content delivery application
27 Main management application
29 User information database
31 DNA information database
33 Content database
35 Charging database
41 Terminal communication unit
43 Control unit
45 Memory unit
47 Display unit
49 Operation system
51 Content data unit

1. A genetic testing and suitability simulation system that is capable of analyzing a gene from DNA of a user and simulating potential suitability of the user, the genetic testing and suitability simulation system comprising:
sampling a cell of the user on which DNA analysis is possible (Step 1);
analyzing DNA arrangement information from the cell sampled at Step 1 (Step 2);
performing genetic analysis from the DNA arrangement information to identify a genetic type (Step 3);
selecting a genetic type suitable for an arbitrary sport motion from an arbitrary genetic polymorphism (Step 4);
comparing the genetic type of the user identified through the genetic analysis with the genetic type selected at Step 4 (Step 5);
calculating a degree of the user's suitability for the arbitrary sport motion from a result of comparison (Step 6);
determining whether the user is suitable for the arbitrary sport motion from the degree of suitability calculated at Step 6 (Step 7); and
presenting a result of calculation at Step 6 and a result of determination at Step 7 to the user as a result of suitability for the arbitrary sport motion through a device possessed by the user (Step 8).

2. A genetic testing and suitability simulation system that is capable of analyzing a gene from DNA of a user and simulating potential suitability of the user, the genetic testing and suitability simulation system comprising:
sampling a cell of the user on which DNA analysis is possible (Step 1);
analyzing DNA arrangement information from the cell sampled at Step 1 (Step 2);
performing genetic analysis from the DNA arrangement information to identify a genetic type (Step 3);
selecting a genetic polymorphism related to an arbitrary sport motion (Step 4);
selecting an arbitrary genetic type from the genetic polymorphism selected at Step 4 (Step 5);
comparing the genetic type of the user identified through the genetic analysis at Step 3 with the genetic type selected from the genetic polymorphism related to the arbitrary sport motion at Step 5 (Step 6);
calculating a degree of the user's suitability for the arbitrary sport motion from a result of comparison (Step 7);
determining whether the user is suitable for the arbitrary sport motion from the degree of suitability calculated at Step 7 (Step 8); and
presenting a result of calculation at Step 7 and a result of determination at Step 8 to the user as a result of suitability for the arbitrary sport motion through a device possessed by the user (Step 9).

3. A genetic testing and suitability simulation system that is capable of analyzing a gene from DNA of a user and simulating potential suitability of the user, the genetic testing and suitability simulation system comprising:
sampling a cell of the user on which DNA analysis is possible (Step 1);
analyzing DNA arrangement information from the cell sampled at Step 1 (Step 2);
performing genetic analysis from the DNA arrangement information to identify a genetic type (Step 3);
selecting a genetic type suitable for an arbitrary game motion from an arbitrary genetic polymorphism (Step 4);
comparing the genetic type of the user identified through the genetic analysis at Step 3 with the genetic type suitable for the arbitrary game motion selected at Step 4 (Step 5);
calculating a degree of the user's suitability for the arbitrary game motion from a result of comparison (Step 6);
determining whether the user is suitable for the arbitrary game motion from the degree of suitability calculated at Step 6 (Step 7); and
presenting a result of calculation at Step 6 and a result of determination at Step 7 to the user as a result of suitability for the arbitrary game motion through a device possessed by the user (Step 8).

4. The genetic testing and suitability simulation system according to claim 1, further comprising presenting a role or a position of the user in an arbitrary sport from the result of suitability for the arbitrary sport motion and the result of suitability for the arbitrary game motion.

5. The genetic testing and suitability simulation system according to claim 2, further comprising presenting a role or a position of the user in an arbitrary sport from the result of suitability for the arbitrary sport motion and the result of suitability for the arbitrary game motion.

6. The genetic testing and suitability simulation system according to claim 3, further comprising presenting a role or a position of the user in an arbitrary sport from the result of suitability for the arbitrary sport motion and the result of suitability for the arbitrary game motion.
